(19) United States
(12) Patent Application Publication
(10) Pub. No.: US 2026/0026765 A1
(43) Pub. Date: Jan. 29, 2026

(54) INTRAORAL SENSOR

(71) Applicants: RAYENCE Co., Ltd., Gyeonggi-do (KR); VATECH EWOO Holdings Co., Ltd., Gyeonggi-do (KR); Qpix solutions Inc., Morrisville, NC (US)

(72) Inventors: Seungman YUN, Morrisville, NC (US); Jung Do Kim, Gyeonggi-do (KR); Jin Sung Lee, Gyeonggi-do (KR); Yun u Ha, Gyeonggi-do (KR)

(73) Assignees: RAYENCE Co., Ltd., Gyeonggi-do (KR); VATECH EWOO Holdings Co., Ltd., Gyeonggi-do (KR); Qpix solutions Inc., Morrisville, NC (US)

(21) Appl. No.: 19/281,440

(22) Filed: Jul. 25, 2025

Related U.S. Application Data
(60) Provisional application No. 63/675,487, filed on Jul. 25, 2024.

(51) Int. Cl. A61B 6/51; A61B 6/42

(52) U.S. Cl. CPC A61B 6/512; A61B 6/425; A61B 2562/18

(57) ABSTRACT

Proposed is an Intraoral sensor. The Intraoral sensor includes a detector module including a sensor assembly in which a photoconductive layer formed of perovskite is formed, a case providing a space in which the detector module is positioned and including at least one metal layer, and a desiccant filling an internal space of the case in a state in which the detector module is accommodated in the case.

### CROSS REFERENCE TO RELATED APPLICATION

This application claims the benefit of the filing date of U.S. Patent Provisional Application No. 63/675,487, filed Jul. 25, 2024, as Attorney Docket No.: 810.0168prov, the teaches of which are incorporated herein their entirety by reference.

### BACKGROUND

### Technical Field

The present disclosure relates to an Intraoral sensor.

### Description of the Related Art

In recent years, a digital detector has become widely used for intraoral X-ray imaging.

An Intraoral sensor may experience performance degradation when moisture and so on penetrate into an inner portion of the Intraoral sensor. In order to prevent such performance degradation, a sealing structure is applied, but there is a problem that the existing sealing structure does not have sufficient moisture prevention characteristics.

### SUMMARY

Accordingly, the present disclosure has been made keeping in mind the above problems occurring in the related art, and an objective of the present disclosure is to provide an Intraoral sensor having a sealing structure capable of effectively preventing moisture penetration.

In order to achieve the objective of the present disclosure, according to an aspect of the present disclosure, there is provided an Intraoral sensor including: a detector module including a sensor assembly in which a photoconductive layer formed of perovskite is formed; a case providing a space in which the detector module is positioned and including at least one metal layer; and a desiccant filling an internal space of the case in a state in which the detector module is accommodated in the case.

The case may include a first case positioned at a front side and a second case positioned at a rear side, and each of the first and second cases may include at least one metal layer.

A thickness of the metal layer may be equal to or more than 0.1 mm and equal to or less than 0.4 mm.

The metal layer may be formed of at least one selected from aluminum, magnesium, and titanium, or may be formed of an alloy thereof.

Each of the first and second cases may further include a plastic layer.

The Intraoral sensor may further include a protective sleeve formed along a side surface of the detector module.

The protective sleeve may include a step portion, and an upper surface of the step portion may be positioned corresponding to a coupling surface where the first case and the second case are coupled to each other.

The Intraoral sensor may further include a sealing material applied to a space of a coupling surface between the first case and the second case.

The sealing material may be formed of a resin including epoxy.

The detector module may be seated on an inner floor surface of the first case, and the desiccant may be positioned at a lateral side and a rear side of the detector module.

The desiccant may have a porous structure.

In the Intraoral sensor according to the present disclosure, the protective sleeve is formed along the side surface of the detector module provided with the photoconductive layer vulnerable to moisture, and the sealing material is applied to the upper surface of the protective sleeve, so that the sealing material is introduced into the space of the coupling surface between the front case and the rear case and the space is filled with the sealing material. Therefore, as the front case and the rear case are solidly coupled to each other by the sealing material, the inner portion of the case may be sealed and the detector module may be coupled to and fixed to the case.

Furthermore, the space inside the case in which the detector module is accommodated may be filled with the desiccant.

As such, since the moisture-proof and dehumidification structure for the photoconductive layer to be prevented from moisture penetration is provided in the Intraoral sensor, moisture penetration into the photoconductive layer may be prevented at a very high level.

Therefore, the Intraoral sensor has the sealing structure that effectively prevents moisture penetration, thereby maximizing the yield rate.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a cross-sectional view schematically illustrating a configuration of an Intraoral sensor according to an embodiment of the present disclosure;

FIG. 2 and FIG. 3 are cross-sectional views schematically illustrating a manufacturing process of the Intraoral sensor according to an embodiment of the present disclosure; and

### DETAILED DESCRIPTION

Hereinafter, an embodiment of the present disclosure will be described in detail with reference to the drawings.

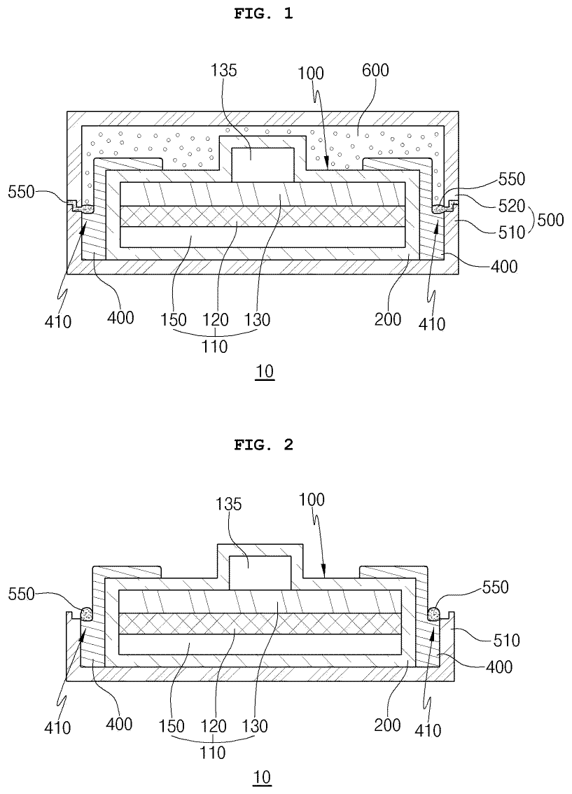

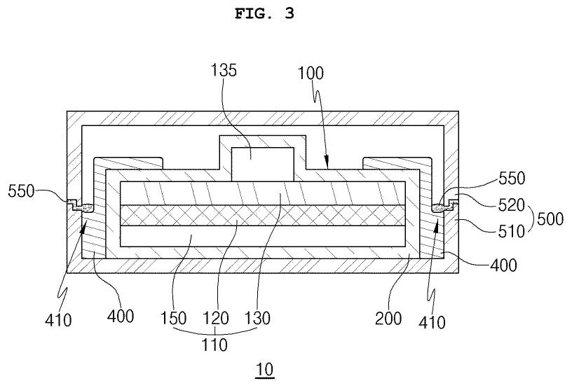

FIG. 1 is a cross-sectional view schematically illustrating a configuration of an Intraoral sensor according to an embodiment of the present disclosure. FIG. 2 and FIG. 3 are cross-sectional views illustrating a manufacturing process of the Intraoral sensor according to an embodiment of the present disclosure, wherein FIG. 2 is a view illustrating a state in which a detector module is seated on a front case and a sealing material is applied on a protective sleeve and FIG. 3 is a view illustrating a state in which a rear case is coupled to the front case.

Referring to FIGS. 1 to 3, the Intraoral sensor 10 according to the present embodiment may be configured as a direct conversion type Intraoral sensor that directly detects incident X-rays and converts the incident X-rays into electrical signals.

The Intraoral sensor 10 may include a detector module (or a sensor module) 100, a protective sleeve 400, and a case (or an outer case) 500.

Here, the detector module 100 may include a sensor assembly 110, and may include a sealing member 200 sealing the sensor assembly 110.

First, the sensor assembly 110 may include a sensor substrate 120, a photoconductive layer (or a photoelectric material layer) 150, and a driving circuit board 130.

As such, the Intraoral sensor 10 of the present embodiment may be provided with the photoconductive layer 150 that directly detects X-rays and generates electrical signals.

Such a photoconductive layer 150 may be formed on a front surface (or an upper surface) of the sensor substrate 120, and may be formed of, for example, perovskite, but is not limited thereto.

Meanwhile, although not specifically illustrated, a lower electrode may be formed for each pixel unit under the photoconductive layer 150. In addition, an upper electrode to which a bias voltage is applied may be formed on the photoconductive layer 150.

Meanwhile, the sensor substrate 120 may be formed of a CMOS substrate or a TFT substrate.

The driving circuit board 130 may be disposed at a rear side of the sensor substrate 120. In other words, the sensor substrate 120 may be positioned on a side opposite to the photoconductive layer 150 with the sensor substrate 120 interposed therebetween. A driving circuit for driving the sensor substrate 120 may be mounted on such a driving circuit board 130.

Meanwhile, a connector 135 for connection with an external system circuit may be mounted on the driving circuit board 130. A cable may be connected to such a connector 135, and electrical connection with the external system circuit may be realized through the cable.

As described above, the sensor assembly 110 of the present embodiment may be configured as a stacked structure in which the driving circuit board 130, the sensor substrate 120, and the photoconductive layer 150 are stacked.

Meanwhile, although not specifically illustrated, the photoconductive layer 150 of the sensor assembly 110 may be configured such that substantially the entire outer surface of the photoconductive layer 150 is covered with a sealant. When the sealant is formed as described above, moisture penetration into an inner portion of photoconductive layer 150 may be prevented.

The sensor assembly 110 configured as described above may be configured to be sealed by the sealing member 200.

In this regard, for example, the sensor assembly 110 may be configured such that substantially the entire outer surface of the sensor assembly 110 is wrapped and sealed by the sealing member 200.

Such a sealing member 200 may include a waterproof material (or a moisture-proof material) such as parylene, silicone, or epoxy, but is not limited thereto.

Meanwhile, the sealing member 200 may be formed as a single layer or multiple layers.

The sealing member 200 and the sensor assembly 110 as described above may constitute the detector module 100.

The detector module 100 may be encased in the case 500.

In this regard, the case 500 may be formed of a front case (or a first case) 510 positioned at a front side to which X-rays are emitted and a rear case (or a second case) 520. The front case 510 and the rear case 520 are coupled to each other, so that an accommodating space may be provided inside the case 500.

Here, the rear case 520 may have, for example, a protrusion in the center portion that protrudes rearward.

The detector module 100 may be inserted into and accommodated in the accommodating space inside the case 500.

In this regard, for example, as illustrated in FIG. 2, the detector module 100 may be seated on an inner floor surface of the front case 510. Then, as illustrated in FIG. 3, the rear case 520 may cover the detector module 100 and may be coupled to the front case 510. More specifically, a front surface of the detector module 100 may be in close contact with the inner floor surface of the front case 510.

Here, in order to couple the front case 510 and the rear case 520 to be solidly fixed to each other and to seal the inner portion of the case 500, the protective sleeve 400 and a sealing material (or a coupling material) 550 may be used.

In this regard, for example, the protective sleeve 400 may be formed such that the protective sleeve 400 surrounds a side surface of the detector module 100. That is, the protective sleeve 400 may be formed along a circumference of the side surface of the detector module 100 and may surround the side surface of the detector module 100. Furthermore, the protective sleeve 400 may be formed such that the protective sleeve 400 may cover at least a portion of the rear side of the detector module 100.

The protective sleeve 400 formed as described above may be formed such that the protective sleeve 400 has a step portion 410 on the side surface of the detector module 100. In this regard, for example, on the side surface of the detector module 100, a lower portion of the protective sleeve 400 may protrude outwardly compared to an upper portion of the protective sleeve 400 so that the step portion 410 having a stepped structure may be formed on the protective sleeve 400.

Such a step portion 410 may be positioned such that the upper surface of the step portion 410 is positioned corresponding to a coupling portion (or a coupling surface) where the front case 510 and the rear case 520 are coupled to each other. More specifically, the upper surface of the step portion 410 may be positioned lower than the position of the coupling portion (or the coupling surface) where the front case 510 and the rear case 520 are coupled to each other.

Here, for example, the protective sleeve 400 may be formed of a resin such as silicone as a waterproof material, but is not limited thereto.

The upper surface of the step portion 410 of such a protective sleeve 400 serves as a seating surface for the sealing material 550, and the sealing material 550 may be applied to the upper surface of the step portion 410.

Here, the sealing material 550 may be formed of a resin including epoxy as a waterproof material, but is not limited thereto.

As illustrated in FIG. 2 and FIG. 3, in a state in which the sealing material 550 is applied to the upper surface of the step portion 410, when the rear case 520 is coupled to the front case 510, the sealing material 550 may be introduced into a space of the coupling surface between the front case 510 and the rear case 520 so that the space is filled with the sealing material 550. After that, by performing a curing process, the sealing material 550 may be cured.

Accordingly, by filling the space of the coupling surface between the front case 510 and the rear case 520 with the sealing material 550, the inner portion of the case 500 may be sealed while the front case 510 and the rear case 520 are coupled to each other.

In addition, since the sealing material 550 is applied and cured such that the sealing material 550 is in contact with the detector module 100, the protective sleeve 400, and the case 500, the detector module 100 may be coupled to and fixed to the case 500.

Meanwhile, the accommodating space inside the case 500 in which the detector module 100 is seated may be filled with a desiccant 600. More specifically, among the accommodating space inside the case 500, a space other than a space where the detector module 100, the protective sleeve 400, and the sealing material 550 are positioned may be filled with the desiccant 600.

In this situation, the desiccant 600 is substantially positioned at the lateral side and the rear side of the detector module 100, and is not positioned at the front side of the detector module 100. Accordingly, the desiccant 600 does not block a light-receiving region (or an active region) of the front side of the sensor assembly 110 of the detector module 100, so that interference with X-ray sensing of the sensor assembly 110 by the desiccant 600 may be prevented.

For example, the desiccant 600 may have a porous structure. When the desiccant 600 has such a porous structure, dehumidification characteristics of the desiccant 600 may be increased.

For example, such a desiccant 600 may be formed of a polymer compound including silica gel, aluminosilicate, calcium sulfate, calcium chloride, and so on, but is not limited thereto.

Meanwhile, the case 500, i.e., each of the front case 510 and the rear case 520, may be configured as a single-layer structure or a multi-layer structure including at least one metal layer.

Here, for example, the metal layer forming the case 500 may include at least one selected from aluminum, magnesium, and titanium, or may include an alloy thereof. As such, when the case 500 is formed such that the case 500 has the metal layer, substantially permanent moisture prevention characteristics may be secured.

For example, a thickness of the metal layer forming the case 500 may be 0.1 mm or more and 0.4 mm or less (i.e., 0.1 mm to 0.4 mm). When the metal layer is formed such that the thickness of the metal layer is within the thickness range as described above, the thickness of the case 500 may be substantially minimized while the moisture prevention characteristics are sufficiently secured.

As described above, in the Intraoral sensor of the present embodiment, the protective sleeve is formed along the side surface of the detector module provided with the photoconductive layer vulnerable to moisture, and the sealing material is applied to the upper surface of the step portion of the protective sleeve, so that the sealing material is introduced into the space of the coupling surface between the front case and the rear case and the space is filled with the sealing material. Therefore, as the front case and the rear case are solidly coupled to each other by the sealing material, the inner portion of the case may be sealed and the detector module may be coupled to and fixed to the case.

Furthermore, the space inside the case in which the detector module is accommodated may be filled with the desiccant.

As such, since the moisture-proof and dehumidification structure for the photoconductive layer to be prevented from moisture penetration is provided in the Intraoral sensor, moisture penetration into the photoconductive layer may be prevented at a very high level.

Therefore, the Intraoral sensor has the sealing structure that effectively prevents moisture penetration, thereby maximizing the yield rate.

The above-described embodiment of the present disclosure is an example of the present disclosure, and free modification is possible within the scope included in the spirit of the present disclosure. Accordingly, the present disclosure includes modifications of the present disclosure within the scope of the appended claims and the equivalents thereof.

What is claimed is:

1. An intraoral sensor comprising:
a detector module comprising a sensor assembly in which a photoconductive layer formed of perovskite is formed;
a case providing a space in which the detector module is positioned and comprising at least one metal layer; and
a desiccant filling an internal space of the case in a state in which the detector module is accommodated in the case,
wherein a thickness of the metal layer is equal to or more than 0.1 mm and equal to or less than 0.4 mm.

2. The intraoral sensor of claim 1, wherein the case comprises a first case positioned at a front side and a second case positioned at a rear side, and each of the first and second cases comprises at least one metal layer.

3. The intraoral sensor of claim 2, wherein the metal layer is formed of at least one selected from aluminum, magnesium, and titanium, or is formed of an alloy thereof.

4. The intraoral sensor of claim 2, further comprising:
a protective sleeve formed along a side surface of the detector module.

5. The intraoral sensor of claim 4, wherein the protective sleeve comprises a step portion, and an upper surface of the step portion is positioned corresponding to a coupling surface where the first case and the second case are coupled to each other.

6. The intraoral sensor of claim 4, further comprising:
a sealing material applied to a space of a coupling surface between the first case and the second case.

7. The intraoral sensor of claim 6, wherein the sealing material is formed of a resin comprising epoxy.

8. The intraoral sensor of claim 2, wherein the detector module is seated on an inner floor surface of the first case, and the desiccant is positioned at a lateral side and a rear side of the detector module.

9. The intraoral sensor of claim 1, wherein the desiccant has a porous structure.
